(19) United States
(12) Patent Application Publication
(10) Pub. No.: US 2019/0133143 A1
(43) Pub. Date: May 9, 2019

(54) FRUIT RIPENING SYSTEM AND FRUIT RIPENING METHOD USING THE SAME

(71) Applicant: INSTITUTE FOR INFORMATION INDUSTRY, Taipei City (TW)

(72) Inventor: Chien-Der Lin, Kaohsiung City (TW)

(73) Assignee: INSTITUTE FOR INFORMATION INDUSTRY, Taipei City (TW)

(21) Appl. No.: 15/828,810

(22) Filed: Dec. 1, 2017

(30) Foreign Application Priority Data
Nov. 7, 2017 (TW) 106138454

(51) Int. Cl. A23B 7/152; A23B 7/04

(52) U.S. Cl. CPC A23B 7/152; C12N 15/8249; A23B 7/04

(57) ABSTRACT

A fruit ripening system and a fruit ripening method using the same are provided. The fruit ripening system includes an image capturing device, a ripening gas generator and a controller. The image capturing device is configured to capture an image of a fruit within a closed container. The ripening gas generator is configured to supply a ripening gas to the closed container. The controller is configured to analyze the image of the fruit to determine an initial maturity of the fruit, determine a required ripening gas concentration according to the initial maturity and a target maturity, and control the ripening gas generator to supply the ripening gas to the closed container for controlling a ripening gas concentration within the closed container at the required ripening gas concentration.

This application claims the benefit of Taiwan application Serial No. 106138454, filed Nov. 7, 2017, the disclosure of which is incorporated by reference herein in its entirety.

### TECHNICAL FIELD

The disclosure relates in general to a fruit ripening system and a fruit ripening method using the same, and more particularly to a fruit ripening system using a ripening gas generator and a fruit ripening method using the same.

### BACKGROUND

Current climacteric fruit, such as bananas, apples, tomatoes and other fruits, are harvested when they are unripe, and then are ripened according to the after-treatment method. For example, the way of ripening banana is to put it in a closed container (such as ripening box, ripening bag, ripening cabinet, or ripening room) with a Celsius temperature of 15 degrees to 20 degrees and then input ripening gas to let the epidermis of the fruit absorb and mature. After a period of time to open the indoor room or the closed container, the fruit maturity may be determined by the human. If the fruit maturity is not enough, it need to continue ripening. However, this approach is entirely dependent on human experience, it is difficult to accurately grasp the progress of fruit ripening.

### SUMMARY

The disclosure is directed to a fruit ripening system and a fruit ripening method using the same capable of improving the above problem.

According to one embodiment, a fruit ripening system is provided. The fruit ripening system includes an image capturing device, a ripening gas generator and a controller. The image capturing device is configured to capture an image of a fruit within a closed container. The ripening gas generator is configured to supply a ripening gas to the closed container. The controller is configured to analyze the image of the fruit to determine an initial maturity of the fruit, determine a required ripening gas concentration according to the initial maturity and a target maturity, and control the ripening gas generator to supply the ripening gas to the closed container for controlling a ripening gas concentration within the closed container at the required ripening gas concentration.

According to another embodiment, a fruit ripening method is provided. The fruit ripening method includes the following steps. An image of a fruit within a closed container is captured; the image of the fruit to determine an initial maturity of the fruit is analyzed; a required ripening gas concentration according to the initial maturity and a target maturity is determined; and the ripening gas generator is controlled to supply the ripening gas to the closed container for controlling a ripening gas concentration within the closed container at the required ripening gas concentration.

### BRIEF DESCRIPTION OF THE DRAWINGS

FIG. 1 illustrates a block diagram of a fruit ripening system;

FIG. 2 illustrates a block diagram of a fruit ripening system according to an embodiment of the present invention;

FIG. 3A illustrates a diagram of a plurality of first relationships between maturities and ripening days according to an embodiment of the present invention;

FIG. 3B illustrates a diagram of a plurality of second relationships between ripening concentrations and ripening days according to an embodiment of the present invention; and

FIG. 4 illustrates a flow chart of a fruit ripening method using the fruit ripening system of FIG. 1.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

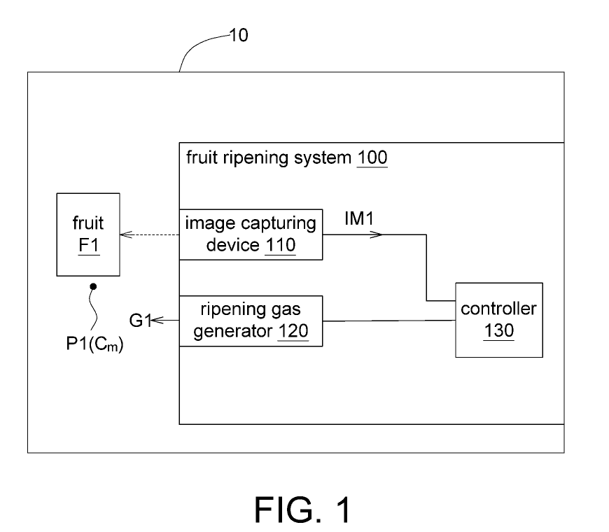

FIG. 1 illustrates a block diagram of a fruit ripening system 100. The fruit ripening system 100 includes an image capturing device 110, a ripening gas generator 120 and a controller 130.

The image capturing device 110 is configured to capture an image IM1 of the fruit F1 within the closed container 10. The ripening gas generator 120 is configured to provide the ripening gas G1 to the closed container 10. The controller 130 is configured to analyze the image IM1 of the fruit F1 to determine the initial maturity M₀ (not illustrated in FIG. 1) of the fruit F1. The controller 130 is further configured to analyze determine a required ripening gas concentration Cᵣ (not illustrated in FIG. 1) according to the initial maturity M₀ and the target maturity Mₜ (not illustrated in FIG. 1), and control the ripening gas generator 120 to supply the ripening gas G1 to the closed container 10 so as to control a ripening gas concentration Cₘ within the closed container 10 at the required ripening gas concentration Cᵣ. In an embodiment, the controller 130 may estimate the required ripening gas G1 according to the capacity of the closed container 10 and the required ripening gas concentration Cᵣ, or set a concentration sensor to detect the ripening gas concentration Cₘ within the closed container 10. In summary, the fruit ripening system 100 of the embodiment of the present invention can automatically adjust the required ripening gas concentration Cᵣ to automatically ripen the fruit F1, and accordingly it may reduce labor cost and make the ripening operation more efficient.

In addition, the embodiment of the present invention is not limited to the type of fruit F1, which may be any fruit which needs to be ripened, such as a more advanced fruit. Color of the fruit may vary according to degree of ripeness. Therefore, the controller 130 may determine the maturity of the fruit F1 by analyzing the color of the fruit of the image IM1 (such as RGB).

At least one of the image capturing device 110 and the ripening gas generator 120 may be electrically connected to the controller 130 through wireless technology or at least one conductive wire. In addition, at least one portion of the fruit ripening system 100 may be located within the closed container 10. For example, the image capturing device 110, the ripening gas generator 120 and the controller 130 are all disposed within the closed container 10. However, the controller 130 may also be disposed outside the closed container 10. In addition, the fruit ripening system 100 may be integrated into a single device, such as a portable device for easy portability and placement in the closed container.

In addition, the image capturing device 110 is, for example, a video camera or photographic camera. The controller 130 may control the image capturing device 110 to capture one image of the fruit F1 at a specific time or continuously capture a plurality of images of the fruit F1 during a certain period of time. The ripening gas generator 120 is, for example, a generator of ethylene gas or other ripening gas. The type of the ripening gas generator 120 may depend on the type of the fruit F1, which is not limited by the embodiment of the present invention. In addition, the ripening gas concentration Cₘ is, for example, the gas concentration at a detection point P1. The detection point P1 is, for example, near the fruit F1 or on the fruit F1 itself, but the embodiment of the present invention is not limited thereto. Since the detection point P1 is, for example, near the fruit F1 or on the fruit F1 itself, the ripening gas concentration Cₘ around the fruit F1 is closer to the required ripening gas concentration Cᵣ, so that ripening of the fruit F1 can be controlled more accurately.

In addition, the embodiment of the present invention does not limit the volume of the closed container 10, which may be a freight container, a closed room, a freezer, a fridge, a cabinet, a box, a bag or other space capable of containing the fruit F1. The size and/or the type of the closed container 10 may be determined according to the number of the fruits F1, which is not limited in the embodiment of the present invention. In addition, the closed container 10 may be movable or fixed.

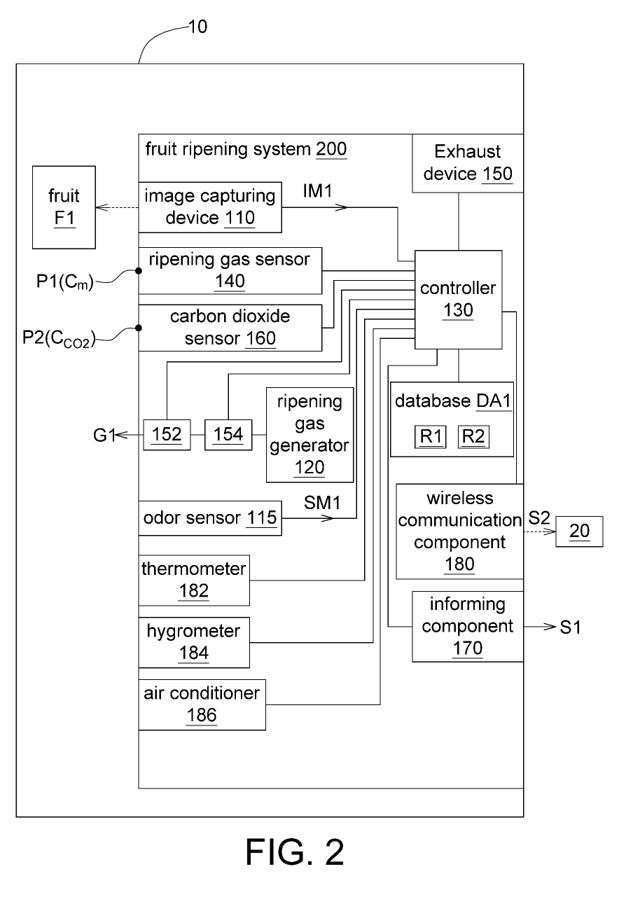

FIG. 2 illustrates a block diagram of a fruit ripening system 200 according to an embodiment of the present invention. The fruit ripening system 200 includes the image capturing device 110, an odor sensor 115, the ripening gas generator 120, the controller 130, a ripening gas sensor 140, an exhaust device 150, a control valve 152, a flow meter 154, a carbon dioxide sensor 160, an informing component 170, a wireless communication component 180, a thermometer 182, a hygrometer 184 and an air conditioner 186.

At least one of the image capturing device 110, the odor sensor 115, the ripening gas generator 120, the ripening gas sensor 140, the exhaust device 150, the control valve 152, the flow meter 154, the carbon dioxide sensor 160, the informing component 170, the wireless communication component 180, the thermometer 182, the hygrometer 184 and the air conditioner 186 may be electrically connected to the controller 130 through a wireless technology or at least one conductive wire. In addition, at least one portion of the fruit ripening system 200 may be located within the closed container 10. For example, the image capturing device 110, the odor sensor 115, the ripening gas generator 120, the ripening gas sensor 140, the exhaust device 150, the carbon dioxide sensor 160, the thermometer 182, the hygrometer 184 and the air conditioner 186 may be disposed in the closed container 10, the others can be disposed outside the closed container 10, or the entire fruit ripening system 200 may also be disposed within the closed container 10.

The odor sensor 115 is disposed within the closed container 10 to sense an odor SM1 of the fruit F1. The controller 130 may determine the initial maturity M₀ of the fruit F1 according to the image IM1 and the odor SM1.

In an embodiment, the determining for the initial maturity M₀ of the fruit F1 is mainly the image IM1, and the odor SM1 is auxiliary. For example, the controller 130 may compare the initial maturity M₀ of the fruit F1 based on the image IM1 with the initial maturity M₀ of the fruit F1 based on the odor SM1. If two determination results match, it means that the determined initial maturity M₀ has high credibility. If the difference between the two determination results is excessively large, the controller 130 mainly determines the result based on the image IM1.

In an embodiment, the odor sensor 115 is, for example, an electronic nose or other commercially available sensor. The odor sensor 115 may digitally display odor intensity and odor level for directly determining the degree of ripeness of the fruit.

The ripening gas G1 generated by the ripening gas generator 120 is supplied to the closed container 10 through the flow meter 154 and the control valve 152. The flow meter 154 may calculate the flow rate of the ripening gas G1 therethrough. The controller 130 may control the control valve 152 to be turned on or off according to quantity of flow of the ripening gas G1 passing through the flow meter 154 and control the ripening gas concentration Cₘ detected by the ripening gas sensor 140. When the control valve 152 is opened, the ripening gas G1 generated by the ripening gas generator 120 enters the closed container 10. When the control valve 152 is closed, the ripening gas G1 generated by the ripening gas generator 120 can't enter the closed container 10.

The ripening gas sensor 140 is disposed within the closed container 10 for detecting the ripening gas concentration Cₘ within the closed container 10. The ripening gas sensor 140 may detect the ripening gas concentration Cₘ at itself location (for example, the detection point P1). The controller 130 analyzes the image IM1 of the fruit F1 to determine the initial maturity M₀ (not illustrated in FIG. 2) of the fruit F1, determines the required ripening gas concentration Cᵣ according to the initial maturity M₀ and the target maturity Mt (not illustrated in FIG. 2), and controls the ripening gas generator 120 to supply the ripening gas G1 to the closed container 10 for controlling the ripening gas concentration Cₘ within the closed container 10 to meet the required ripening gas concentration Cᵣ. When the ripening gas concentration Cₘ within the closed container 10 is lower than the required ripening gas concentration Cᵣ, in response to this situation, the controller 130 may control the ripening gas generator 1200 to generate more ripening gas G1 to the closed container 10 so as to allow the ripening gas concentration Cₘ within the closed container 10 to rapidly meet the required ripening gas concentration Cᵣ.

The exhaust device 150 is disposed within the closed container 10 to discharge the gas from the closed container 10 out of the closed container 10 to reduce the ripening gas concentration Cₘ within the closed container 10. In an embodiment, when the ripening gas concentration Cₘ within the closed container 10 is higher than the required ripening gas concentration Cᵣ, in response to this situation, the controller 130 controls the exhaust device 150 to discharge the gas from the closed container 10 while selectively supplies general air or a specific gas to the closed container 10 for reducing the ripening gas concentration Cₘ within the closed container 10 so as to control the ripening gas concentration Cₘ to meet the required ripening gas concentration Cᵣ. In an embodiment, a first upper limit of the ripening gas concentration Cₘ is, for example, 2000 ppm, or may also be less than or greater than 2000 ppm. The controller 130 controls the exhaust device 150 to discharge the gas from the closed container 10 to reduce the ripening gas concentration Cₘ within the closed container 10 when the ripening gas concentration Cₘ within the closed container 10 is higher than the first upper limit value.

Although not illustrated, the fruit ripening system 200 further includes a gas disturbing component. The gas disturbing component is disposed on the closed container 10 to homogenize the gas within the closed container 10 so as to avoid Inhomogeneous distribution of the ripening gas or other kinds of gas within the closed container 10. In addition, the gas disturbing component is, for example, a fan or an air blower. In addition, the gas disturbing component may have a ventilation and/or air blowing function.

In addition, the exhaust device 150 does not aimed at certain or specific gas within the closed container 10, and it may discharge air, the ripening gas G1, the carbon dioxide and other gases in the closed container 10. The controller 130 controls the ripening gas sensor 140 to detect the ripening gas concentration Cₘ within the closed container 10 while the exhaust device 150 discharges the gas from the closed container 10. When the ripening gas concentration Cₘ within the closed container 10 meets the required ripening gas concentration Cᵣ, in response to this situation, the controller 130 controls the exhausting device 150 to stop discharging. In an embodiment, the exhaust device 150 is an exhaust fan, for example.

The ripening process of breathing of the fruit F1 will produce carbon dioxide. The carbon dioxide sensor 160 is disposed within the closed container 10. The carbon dioxide sensor 160 is configured to detect the carbon dioxide concentration C_{CO2} within the closed container 10. The carbon dioxide sensor 160 may be disposed on a detection point P2. The detection point P2 is, for example, near the fruit F1 or on the fruit F1 itself, but the embodiment of the present invention is not limited thereto. In addition, the detecting point P2 may be close to or away from the detecting point P1, but the embodiment of the present invention is not limited thereto.

In one embodiment, when the carbon dioxide concentration C_{CO2} within the closed container 10 is higher than a second upper limit value, in response to this situation, the controller 130 controls the exhaust device 150 to discharge the gas from the closed container 10 so as to reduce the carbon dioxide concentration C_{CO2} within the closed container 10. The second upper limit here is, for example, 3000 ppm. The carbon dioxide concentration C_{CO2} has the effect upon inhibiting ethylene. Since the embodiment of the present invention can timely discharge excess carbon dioxide, the ripening of the fruit may be prevented from being excessively suppressed, and the ripening of the fruit F1 may be controlled more accurately. In addition, the second upper limit value may be inputted by a user through a user interface (not illustrated), or the second upper limit value may be a default value.

In addition, the exhaust device 150 does not aim at certain or specific gas within the closed container 10. The controller 130 controls the carbon dioxide sensor 160 to detect the carbon dioxide concentration C_{CO2} within the closed container 10 while discharging the gas from the closed container 10. When the carbon dioxide concentration C_{CO2} within the closed container 10 is lower than the second upper limit value, in response to this situation, the controller 130 controls the exhaust device 150 to stop discharging.

The informing component 170 may output the first notification signal S1. For example, when the maturity of the fruit F1 matches the target maturity Mₜ, in response to this situation, the controller 130 controls the informing component 170 to output the first notification signal S1 to notify the user that the fruit F1 has been ripened completely. In an embodiment, the informing component 170 is, for example, a light emitter, a sound generator, a vibrator or other element that may output notification signal. In addition, the first notification signal S1 is, for example, color light, sound or vibration.

In an embodiment, when the maturity of the fruit F1 matches the target maturity Mₜ, in response to this situation, the controller 130 may control the wireless communication component 180 to output the second notification signal S2 to the external electronic device 20. The external electronic device 20 is, for example, a portable electronic device, a handheld electronic device, a personal computer, a server, etc. As a result, the user does not need to pay attention to the closed container 10, and only need to passively wait for the automatic notification of the fruit ripening system 100.

In addition, the thermometer 182 and the hygrometer 184 may detect the temperature and the humidity within the closed container 10. The controller 130 controls the air conditioner 186 to adjust the temperature and humidity within the closed container 10 to a suitable temperature value and humidity value according to the measured temperature value by the thermometer 182 and humidity value by the hygrometer 184. Under the control of temperature and/or humidity, the fruit ripening system 200 may accurately ripen the fruit F1. Different fruit F1 need different ripening temperature and ripening humidity. The suitable ripening temperature values and the suitable humidity values for different fruits are recorded in a database DA1. The controller 130 may determine the type of the fruit F1 according to analysis of the image IM1 and control the air conditioner 186 to adjust the temperature and the humidity within the closed container 10 to a temperature and a humidity suitable for ripening the fruit F1 according to the suitable ripening temperature and the suitable humidity recorded in the database DA1. In another embodiment, the thermometer 182, the hygrometer 184 and the air conditioner 186 may also be selectively omitted.

The following is a description of a method for determining the required ripening gas concentration Cᵣ according to an embodiment of the present invention.

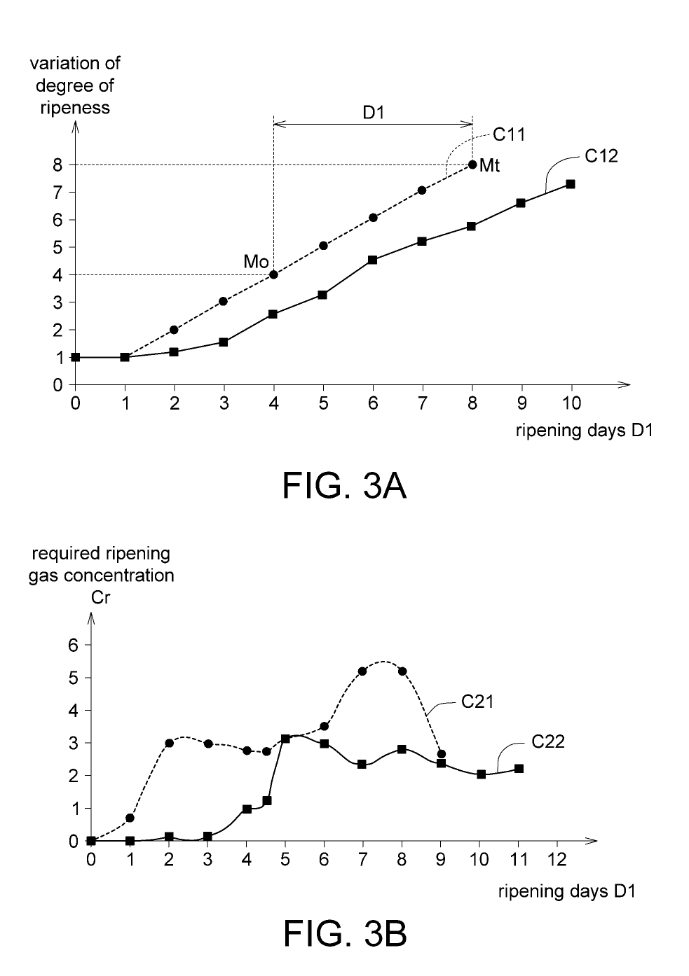

Referring to FIGS. 2, 3A and 3B, FIG. 3A illustrates a diagram of a plurality of first relationships R1 between maturities and ripening days according to an embodiment of the present invention, and FIG. 3B illustrates a diagram of a plurality of second relationships R2 between ripening concentrations and ripening days according to an embodiment of the present invention. As illustrated in FIG. 3A, the horizontal axis indicates the ripening days D1, and the vertical axis indicates the variation of degree of ripeness. The ripeness level of the fruit F1 may be divided into eight levels, and the number of the levels may also be less than or greater than eight. As illustrated in FIG. 3B, the horizontal axis indicates the ripening days D1, and the vertical axis indicates the required ripening gas concentration Cᵣ.

As illustrated in FIG. 2, the database DA1 stores the first relationships R1 (only one is illustrated in FIG. 2) and the second relationships R2 (only one is illustrated in FIG. 2). Each first relationship R1 includes a relationship between the ripening days and the variation of degree of ripeness of the fruit. As illustrated in FIG. 3A, the curves C11 and C12 represent different first relationships R1 of the fruit F1. For example, the curve C11 is a quicker ripening curve and the curve C12 is a slower ripening curve. Each second relationship R2 controls (or includes) the ripening days and the required ripening gas concentration Cᵣ of the fruit F1. As illustrated in FIG. 3B, the curve C21 corresponds to the curve 11, and the curve C22 corresponds to the curve 12. For example, the curve C11 is a quicker ripening curve and the curve C12 is a slower ripening curve. FIGS. 3A and 3B illustrate two curves. However, the number of the curves may be more than two and established according to different ripening days. In addition, other different types of fruit may also establish several different first relationships R1 and the corresponding second relationships R2.

In the present embodiment, the controller 130 determines the first relationship R1 according to the initial maturity M₀, the target maturity Mₜ and the ripening days Dl. For example, the controller 130 determines one first relationship R1 satisfying the initial maturity M₀, the target maturity Mₜ and the ripening days D1 from a plurality of the first relationships R1. Then, the controller 130 determines one second relationship R2 corresponding to the satisfied first relationship R1.

In details, as illustrated in FIG. 3A, if it is desired that the initial maturity M₀ being 4 is changed to the target maturity Mₜ being 8 within 4 days (the ripening days D1), the controller 130 determines one first relationship R1 from a plurality of the first relationships R1, for example, the curve C11 of FIG. 3A. Then, according to the curve C11, the controller 130 determines one second relationship R2 from a plurality of the second relationships R2, for example, the curve C21 of FIG. 3B. The controller 130 may determine the required ripening gas concentration Cᵣ of each day from the curve C21 showing the second relationships R2.

In addition, the aforementioned initial maturity M₀, the target maturity Mₜ and the ripening days D1 are, for example, inputted by a user through a user interface (not illustrated) and then transmitted to the controller 130 via at least one conductive wired or wireless network. Then, the controller 130 determines the first relationship R1 according to input information by the user. Alternatively, the controller 130 may determine the initial maturity M₀ of the fruit F1 according to the image IM1 of the fruit F1, and determine the first relationship R1 according to the initial maturity M₀, the target maturity Mₜ and the ripening days D1, wherein the target maturity Mₜ and ripening days D1 may be default values or the controller 130 automatically determines based on historical data.

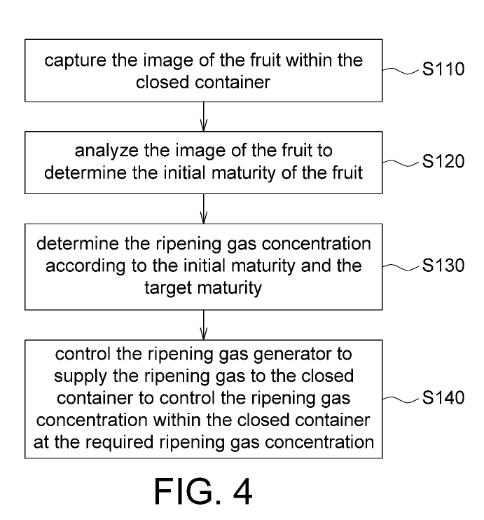

FIG. 4 illustrates a flow chart of a fruit ripening method using the fruit ripening system 100 of FIG. 1.

In step S110, the image capturing device 110 captures the image IM1 of the fruit F1 within the closed container 10. In step S120, the controller 130 analyzes the image IM1 of the fruit F1 to determine the initial maturity M₀ of the fruit F1. In step S130, the controller 130 determines the ripening gas concentration Cₘ within the closed container 10 according to the initial maturity M₀ and the target maturity Mₜ. In step S140, the controller 130 controls the ripening gas generator 120 to supply the ripening gas G1 to the closed container 10 to control the ripening gas concentration Cₘ within the closed container 10 at the required ripening gas concentration Cᵣ. In an embodiment, the steps S110 to S140 are performed sequentially every a designated period of time, and the designated period of time is, for example, one day, or several hours.

In addition, the fruit ripening method of the fruit ripening system 200 may refer to the aforementioned description of FIG. 2, and details are not described herein.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A fruit ripening system, comprising:
an image capturing device configured to capture an image of a fruit within a closed container;
a ripening gas generator configured to supply a ripening gas to the closed container; and
a controller configured to:
analyze the image of the fruit to determine an initial maturity of the fruit;
determine a required ripening gas concentration according to the initial maturity and a target maturity; and
control the ripening gas generator to supply the ripening gas to the closed container for controlling a ripening gas concentration within the closed container at the required ripening gas concentration.

2. The fruit ripening system according to claim 1, further comprising:
a ripening gas sensor disposed within the closed container for detecting the ripening gas concentration within the closed container.

3. The fruit ripening system according to claim 1, further comprising:
an exhaust device disposed within the closed container;
wherein the controller is further configured to:
in response to the ripening gas concentration within the closed container being higher than the required ripening gas concentration, control the exhaust device to discharge gas from the closed container so as to reduce the ripening gas concentration within the closed container.

4. The fruit ripening system according to claim 1, wherein the controller is configured to:
in response to the ripening gas concentration within the closed container being lower than the required ripening gas concentration, control the ripening gas generator to supply more ripening gas to the closed container.

5. The fruit ripening system according to claim 1, wherein for determining the required ripening gas concentration according to the initial maturity and the target maturity, the controller is configured to:
determine a first relationship between maturities and ripening days according to the initial maturity, the target maturity and ripening days; and
determine a second relationship between ripening concentrations and ripening days according to the first relationship, wherein the second relationship controls the required ripening gas concentration of every day during the ripening days.

6. The fruit ripening system according to claim 5, further comprising a database storing a plurality of the first relationships and a plurality of the second relationships; for determining the first relationship according to the initial maturity, the target maturity and the ripening days, the controller is configured to:
determine one of the first relationships which satisfies the initial maturity, the target maturity and the ripening days from the plurality of the first relationships; and
wherein for determining the second relationship according to the first relationship, the second relationship corresponding to the satisfied first relationship is determined from the plurality of the second relationships.

7. The fruit ripening system according to claim 1, further comprising an exhaust device and a carbon dioxide sensor, and the exhaust device and the carbon dioxide sensor are disposed within the closed container, wherein the carbon dioxide sensor is configured to detect a carbon dioxide concentration within the closed container, and the controller is further configured to:
in response to the carbon dioxide concentration within the closed container being higher than an upper limit value, control the exhaust device to discharge gas from the closed container so as to reduce the carbon dioxide concentration within the closed container.

8. The fruit ripening system according to claim 1, further comprising an informing component, and the controller is further configured to:
in response to the maturity of the fruit matching the target maturity, control the informing component to output a first informing signal.

9. The fruit ripening system according to claim 1, further comprising a wireless communication component, and the controller is further configured to:
in response to the maturity of the fruit matching the target maturity, control the wireless communication component to output a second informing signal to an external electronic device.

10. The fruit ripening system according to claim 1, further comprising an odor sensor disposed within the closed container for sensing an odor of the fruit, and the controller is further configured to:
analyze the odor to determine the initial maturity of the fruit according to the odor and the image.

11. A fruit ripening method, comprising:
capturing an image of a fruit within a closed container;
analyzing the image of the fruit to determine an initial maturity of the fruit;
determining a required ripening gas concentration according to the initial maturity and a target maturity; and
controlling the ripening gas generator to supply the ripening gas to the closed container for controlling a ripening gas concentration within the closed container at the required ripening gas concentration.

12. The fruit ripening method according to claim 11, wherein step of capturing the image of the fruit within the closed container, step of analyzing the image of the fruit to determine the initial maturity of the fruit and step of determining the required ripening gas concentration according to the initial maturity and the target maturity are performed every a designated period of time.

13. The fruit ripening method according to claim 11, further comprising a ripening gas sensor disposed within the closed container for detecting the ripening gas concentration within the closed container.

14. The fruit ripening method according to claim 11, further comprising:
in response to the ripening gas concentration within the closed container being higher than the required ripening gas concentration, discharging gas from the closed container so as to reduce the ripening gas concentration within the closed container.

15. The fruit ripening method according to claim 11, further comprising:
in response to the ripening gas concentration within the closed container being lower than the required ripening gas concentration, controlling the ripening gas generator to supply more ripening gas to the closed container.

16. The fruit ripening method according to claim 11, wherein step of determining the required ripening gas concentration according to the initial maturity and the target maturity further comprises:
determining a first relationship between maturities and ripening days according to the initial maturity, the target maturity and ripening days; and
determining a second relationship between ripening concentrations and ripening days according to the first relationship, wherein the second relationship controls the required ripening gas concentration of each day during the ripening days.

17. The fruit ripening method according to claim 16, wherein step of determining the first relationship according to the initial maturity, the target maturity and the ripening days further comprises:
determining the first relationship which satisfies the initial maturity, the target maturity and the ripening days from the plurality of the first relationships; and
wherein for determining the second relationship according to the first relationship, the second relationship corresponding to the satisfied first relationship is determined from the plurality of the second relationships.

18. The fruit ripening method according to claim 11, further comprising:
in response to a carbon dioxide concentration within the closed container being higher than an upper limit value, controlling the exhaust device to discharge the gas from the closed container so as to reduce the carbon dioxide concentration within the closed container.

19. The fruit ripening method according to claim 11, further comprising:
in response to the maturity of the fruit matching the target maturity, controlling the informing component to output a first informing signal.

20. The fruit ripening method according to claim 11, further comprising:
sensing an odor of the fruit; and
analyzing the odor of the fruit to determine the initial maturity of the fruit according to the odor and the image.
